(19) United States
(12) Patent Application Publication
(10) Pub. No.: US 2026/0294794 A1
(43) Pub. Date: Oct. 1, 2026

(54) WATER-SOLUBLE BIOMOLECULAR GLASS MICRONEEDLE AND PREPARATION METHOD THEREOF

(71) Applicant: INSTITUTE OF PROCESS ENGINEERING, CHINESE ACADEMY OF SCIENCES, Beijing (CN)

(72) Inventors: Xuehai YAN, Beijing (CN); Ruirui XING, Beijing (CN)

(21) Appl. No.: 19/480,563

(22) PCT Filed: May 5, 2023
(86) PCT No.: PCT/CN2023/092309

§ 371 Date: Oct. 31, 2025

(51) Int. Cl. A61K 9/00; A61K 31/198; A61K 31/4172; A61K 31/519; A61K 38/07; A61K 38/08; A61K 38/13; A61K 38/14; A61K 38/26; A61K 38/28

(52) U.S. Cl. CPC A61K 9/0021; A61K 31/198; A61K 31/4172; A61K 31/519; A61K 38/07; A61K 38/08; A61K 38/13; A61K 38/14; A61K 38/26; A61K 38/28

(57) ABSTRACT

The present disclosure relates to a soluble biomolecular glass microneedle and a method thereof. The microneedle includes a microneedle array and a substrate; where at least a part of the microneedle array consists of a biomolecular glass; and the biomolecular glass consists of a biomolecule, an inducer, and a solvent in a trace amount. With good mechanical strength, the microneedle in the present disclosure can completely and smoothly pierce into skin and mucosa, adjust and quickly release the biomolecule, and effectively improve bioavailability of the biomolecule. Needle tip of the microneedle completely consists of the biomolecule, and can be completely absorbed by the body after release is completed, without increasing metabolic burden in vivo.

### TECHNICAL FIELD

The present disclosure relates to a soluble biomolecular glass microneedle, a preparation method, and use thereof, and belongs to the technical field of a pharmaceutical formulation and a preparation method thereof.

### BACKGROUND

Percutaneous absorption and transdermal delivery have a long history, dating back to the ancient Roman times. Technologies in the art are continuously innovating, such as chemical permeability enhancers, iontophoresis, microdermabrasion, ultrasonic cavitation, and microneedling. In recent years, due to its unique advantages and application potential, microneedling has been selected jointly by Scientific American and the World Economic Forum as one of the top ten emerging technologies of 2020 in the world.

Microneedles are micron-sized (<1000 μm in length) conical, pyramidal, or multifaceted piercing protrusions which offer many advantages for intradermal delivery. Application of microneedles creates temporary channels in the outer layer of the skin, thus bypassing the barrier functionality and allowing the delivery of different bioactive molecules, particularly biomacromolecules such as polypeptides, nucleic acids, and proteins, or stem cells, which otherwise would be incapable of delivery via the transdermal route. The short shaft of the microneedle is long enough to penetrate the stratum corneum but does not penetrate far enough to reach underlying nerve endings. Therefore, the application of microneedles is essentially painless. This increases patient compliance, particularly for those with trypanophobia.

Microneedles are classified into soluble microneedles and insoluble microneedles based on solubility after the needle tips are inserted into the stratum corneum of the skin. Most insoluble microneedles consist of metal, monocrystal silicon, or a cross-linked polymer. To facilitate the transdermal delivery of bioactive molecules, smear or application of the bioactive molecules is required after microneedle removal to allow the bioactive molecules to pass through skin pores formed by the microneedles. Micropores on the skin begin to heal upon microneedle removal. Duration of the microneedle pores is limited by self-repair of the skin, making it difficult to further improve the overall efficiency of transdermal absorption of the bioactive molecules. Furthermore, there is also a risk that the needle tip is broken and becomes lodged in the skin. Soluble microneedles are commonly composed of biocompatible polymers, including water-soluble polymers such as hyaluronic acid, chitosan, polyvinyl alcohol, chondroitin sulfate, polyvinylpyrrolidone, trehalose, dextran, maltose, sucrose, and carboxymethylcellulose. These polymer tip matrices can load bioactive molecules and increase the drug-loading capacity of microneedles, thereby becoming a most promising type of microneedles. For example, a reference literature (Chinese patent CN104027324B) provides a soluble microneedle vaccine patch made of a water-soluble polymer material as a host material and adapted to transdermal delivery of a vaccine, and achieves effective combined use of the vaccine and an adjuvant in a soluble microneedle, thereby improving the effect of the Th1-type immune response, and obtaining relatively balanced Th1- and Th2-type immune response. US patent US2023015942 A1 discloses a microneedle delivery device with a detachable hybrid microneedle depot for delivering mesenchymal stem cells to various tissues and organs for tissue regeneration. Invention patent CN110769812A describes a microneedle system based on polyvinylpyrrolidone (PVP) loading to release glucagon-like peptide analogs. US patent U.S. Pat. No. 9,320,878B2 discloses a polymer microneedle patch that can achieve transdermal controlled release of hydrophilic macromolecules such as proteins, polypeptides, DNA, RNA and other drugs.

The above disclosed soluble microneedles and/or microneedle devices that deliver biomolecules have in common that needle bodies of the microneedles mainly comprise an inactive excipient, which only serves as a carrier of the biomolecules or a plasticizer for the microneedle, so that at present, use of the soluble microneedles is still faced with some to-be-solved problems: on the one hand, before clinical application, it is necessary to study metabolism and elimination pathways of intradermally soluble components to determine the biosafety of the materials, thereby inevitably delaying the commercialization process. Therefore, at present, microneedle devices in clinical trials and clinically approved microneedle devices are still mainly insoluble microneedles. On the other hand, in microneedle applications, existing polymer needle tips may be partially deposited intradermally after being dissolved in vivo, thereby forming granulations, generating local erythema, or accumulating in intracorporeal organs, and resulting in material accumulation or material metabolism burden. In addition, due to small size, microneedles can only deliver a limited number of active molecules, thereby limiting use of the microneedles when a large dose or continuous release of bioactive molecules is required.

In summary, the development of soluble microneedles that do not rely on excipients such as water-soluble polymers and are composed of bioactive molecules themselves can enhance drug delivery capacity and safety of the microneedles, thereby laying the groundwork for broader clinical applications.

### SUMMARY

In order to solve the problems existing in the background, the present disclosure provides a soluble biomolecular glass microneedle and a preparation method thereof. A general strategy is successfully provided to introduce multiple non-covalent bonds and a solvent in a trace amount to obtain a biomolecular glass with processability, and use it to prepare a biomolecular glass microneedle. By means of the aforementioned technical means of kinetic stabilization, the resulting biomolecular glass network has very strong compressibility, shows high mechanical strength, and exhibits no fracture under 90% strain.

As can be seen from existing literature, the salt form, crystal form, crystalline state, and crystallization process of an Active Pharmaceutical Ingredient have significant influence on its stability, solubility, dissolution rate, bioavailability, absorption in vivo, and distribution properties. Therefore, it is necessary to conduct various solid-state development on the Active Pharmaceutical Ingredient to improve its properties, such as characteristic dissolution, solubility, hygroscopicity, and stability. CN113754556B and CN114014908A each disclose a method for preparing an amino acid-based biomolecular glass, a cyclopeptide glass, and a cyclopeptide-containing pharmaceutical composition glass via a melt-quenching process, thereby expanding the application range of these biomolecules.

The needle tip of the microneedle in the present disclosure, which is composed of a biomolecular glass, not only can completely and smoothly pierce into skin and mucosa, but also can modulate the release rate of bioactive molecules, effectively increase the dissolution rate of the bioactive molecules, and improve the bioavailability of the drug. The needle tip of the microneedle completely consists of the biomolecules, and can be completely absorbed by the body after administration, without increasing metabolic burden in vivo. The microneedle of the present disclosure features a high loading capacity for bioactive molecules, a simple and rapid preparation method, and is suitable for industrial production.

In the first aspect, a soluble biomolecular glass microneedle is provided, which includes a microneedle array and a substrate;
where at least a part of the microneedle array consists of a biomolecular glass;
the biomolecular glass consists of a biomolecule, an inducer, and a solvent in a trace amount; the biomolecule includes an amino acid and a derivative and/or a peptide thereof; the solvent in the trace amount refers to a solvent content in the biomolecular glass of 0.1 wt % to 5 wt %, preferably 0.1 wt % to 2 wt %; the inducer is selected from the group consisting of a nucleotide, a nucleotide polymer, RNA, DNA, and/or a pH regulator.

In the second aspect, a soluble biomolecular glass microneedle is provided, where the biomolecular glass refers to an amorphous (non-crystalline) biomolecular matrix that forms a thermodynamically stable liquid, that is, a liquid with extremely high viscosity, corresponding to a physical solid.

The biomolecular glass is glass in a broad sense, and refers to a non-crystalline solid structure composed of biomolecules with short-range order and long-range disorder, and with a glass transition phenomenon (Tg>0° C.).

Preferably, the Tg of the biomolecular glass is in a range of 0° C.<Tg<200° C.

More preferably, the Tg of the biomolecular glass is in a range of 20° C.<Tg<160° C.

The biomolecular glass has a good glass-forming ability (GFA), that is, a ratio of the Tg (glass transition temperature)/Tm (melting temperature) ranges from 0.55 to 0.75, preferably 0.66 to 0.75.

In the third aspect, a soluble biomolecular glass microneedle is provided, where the biomolecular glass consists of a biomolecule, an inducer, and a solvent in a trace amount.

The biomolecule is an amino acid and a derivative and/or a peptide-therapeutic agent thereof.

The amino acid includes one or more of glycine, alanine, valine, leucine, isoleucine, methionine (Met), proline, tryptophan, serine, tyrosine, cysteine, phenylalanine, asparagine, glutamine, threonine, aspartic acid, glutamic acid, lysine, arginine, histidine, selenocysteine, and pyrrolysine.

The derivative is an amino acid or peptide with a protecting group, where the protecting group includes Trt, Boc, Fmoc, Cbz/Z, Allyl, C2-C18 acyl, benzoyl, naphthoyl, OFm, Otbu, OBzl, OAll, OMe, and OEt.

Preferably, the peptide-therapeutic agent has biological and/or pharmacological activity; and the peptide-therapeutic agent may be a linear peptide, a cyclopeptide, or a peptide derivative, or a conjugate.

The peptide-therapeutic agent includes an anti-tumor peptide, a cytokine mimetic peptide, a cardiovascular peptide, a host defense peptide, an immunoregulation peptide, a metabolic peptide, an antiviral polypeptide, and a diagnostic polypeptide.

Exemplary peptide-therapeutic agents include one or more of octreotide acetate, lanreotide acetate, tetracosactide acetate, leuprorelin, buserelin, goserelin, gonadorelin, nafarelin, triptorelin, histrelin, triptorelin, degarelin, abarelix, octreotide, lutathera, somatostatin, lanreotide, pasireotide, romidepsin, mifamurtide, recombinant human insulin, thymopentin, recombinant human interferon, glutathione, teriparatide acetate, semaglutide, liraglutide, lixisenatide, exenatide, benaglutide, dulaglutide, albiglutide, thymopentin, thymalfasin, salcatonin, teriparatide, bacitracin, octreotide, carperitide, neuropeptide Y, brain natriuretic peptide (BNP), islet amyloid polypeptide (IAPP), vasoactive intestinal peptide (VIP), nesiritide, integrelin, oxytocin, bivalirudin, antiphagocytic peptide, vancomycin, methotrexate, and cyclosporine.

The inducer can be introduced to modulate multiple non-covalent interactions among biomolecules.

Preferably, the inducer is selected from the group consisting of a nucleotide, a nucleotide polymer, RNA, DNA, and/or a pH regulator.

Exemplary nucleotides include, but are not limited to, adenosine-5′-monophosphate (AMP), nicotinamide monophosphate, guanosine-5′-monophosphate (GMP), uridine-5′-monophosphate (UMP), cytidine-5′-monophosphate (CMP), and adenosine-3′-monophosphate (3-AMP).

The pH regulator is selected from the group consisting of DL-tartaric acid, hydrochloric acid, sulfuric acid, phosphoric acid, lactic acid, lactobionic acid, citric acid, tartaric acid, oxalic acid, DL-malic acid, maleic acid, quinic acid, adipic acid, fumaric acid, hexanoic acid, heptanoic acid, octanoic acid, valeric acid, butyric acid, propionic acid, and glacial acetic acid, preferably citric acid, nucleotide, and malic acid.

The solvent is water or normal saline. The mass percentage of the solvent is 0 to 5%, preferably 0 to 2%.

In the fourth aspect, a soluble biomolecular glass microneedle is provided, where the biomolecular glass microneedle has good mechanical properties and processability. Considering the skin permeability of the soluble glass microneedle, the needle tip of the glass microneedle has a hardness greater than 50 MPa, and an elastic modulus greater than 1 Gpa, thereby facilitating smoothly piercing skin.

In the fifth aspect, a soluble biomolecular glass microneedle is provided, where the substrate of the soluble biomolecular glass microneedle can be made from a biomolecular glass and/or a polymer excipient.

The polymer excipient is one or more of hydroxypropylcellulose, alginic acid, methylcellulose, chitosan, tragacanth, propylene glycol alginate, maltose, hyaluronic acid, chitosan, polyvinyl alcohol, chondroitin sulfate, polyvinylpyrrolidone, trehalose, dextran, polylactic acid, carrageenan, cellulose acetate, hydroxyethylmethylcellulose, ethylhydroxycellulose, sucrose, carboxymethylcellulose, poly-γ-glutamic acid, pullulan, gelatin, polydopamine, and polyacrylamide.

In the sixth aspect, a preparation method of a soluble biomolecular glass microneedle is provided, where the preparation method includes steps of:
(1) preparing the biomolecular glass by hydrothermal method: dissolving a certain amount of a biomolecular raw material in water or a mixed solvent, adding an appropriate amount of an inducer, adjusting pH of a resulting solution system; maintaining the system at a constant temperature by hydrothermal method, and adjusting a duration of the constant temperature to volatilize the solvent stepwise, so as to obtain the biomolecular glass;
(2) preheating the biomolecular glass, then preparing a microneedle array by casting method, stretching method, atomization spraying method, microfluidics, or 3D printing; and
(3) finally, casting and degassing on the basis of the microneedle array to form a microneedle substrate.

In the above step (1), the system is adjusted to a pH of 1 to 9, preferably to a pH of 3 to 7; the constant temperature is 20° C. to 120° C., preferably 40° C. to 100° C.;
the duration of the constant temperature is 5 min to 6 h, preferably 30 min to 3 h;
a preheating temperature is 20° C. to 200° C., preferably 40° C. to 160° C., more preferably 40° C. to 100° C.

In the seventh aspect, in some embodiments, a process of preparing a biomolecular glass by hydrothermal method can be combined with a process of preparing a microneedle by casting method, that is, preparing through the following steps:
(1) preparing a precasting solution for the microneedle: dissolving a biomolecular raw material in water or a mixed solvent, adding an appropriate amount of an inducer, adjusting pH of a resulting solution system;
(2) pouring the precasting solution into a microneedle mold, and degassing the solution; and
(3) obtaining the microneedle after drying and demolding.

The pouring method includes, but is not limited to, self-leveling, high-pressure injection, and microfluidics.

The method of degassing includes, but is not limited to, centrifugation, depressurization, or vacuum adsorption.

The drying in the step (3) is performed under conditions of: a temperature of 20° C. to 100° C., a relative humidity of 1% to 60%, and a drying time of more than 2 h; further preferably, 20° C. to 50° C., relative humidity of 1% to 30%, and a drying time of more than 2 h.

In the eighth aspect, when the substrate of a resulting microneedle is a polymer excipient, the preparation step (3) of the soluble biomolecular glass microneedle prepared as described in the sixth aspect is improved as follows:
(3) preparing a polymer excipient solution and casting it onto the biomolecular glass microneedle array prepared in the step (2) to form the microneedle substrate.

In the ninth aspect, the preparation method of a soluble biomolecular glass microneedle according to the seventh and eighth aspects can also be combined with other conventional well-known preparation processes to prepare a multi-layer microneedle, a bubble microneedle, and a porous microneedle, and the like.

Physical shape and size of the soluble biomolecular glass microneedle in the present disclosure are not particularly limited, and any size known in the art can be used.

For example, a height of the needle body of the soluble microneedle can be 100-1,000 microns, and an angle of the needle tip can be 30-40 degrees.

The soluble biomolecular glass microneedle, after drying and stripping, may be further cut into a patch shape, and/or further backed with an adhesive support for use.

In the tenth aspect, the soluble biomolecular glass microneedle as described above can be loaded with an additional active substance in the preparation process, the active substance can be loaded on the microneedle array or/and the substrate of the microneedle; when the active substance is loaded on the microneedle array, its mass proportion is less than 5% of the microneedle array; and when the active substance is loaded on the microneedle array and the substrate, its mass proportion is less than 10% of the total mass.

The active substance includes, but is not limited to, a micromolecular drug such as an analgesic drug, a neurological drug, an anti-allergic drug, an anti-anxiety drug, and an anti-inflammatory drug, and a biomacromolecular drug such as an antigen peptide, a peptide vaccine, and a monoclonal antibody. Example micromolecular drugs include lidocaine, fentanyl, aspirin, ibuprofen, cetirizine, loratadine, rofecoxib, celecoxib, diclofenac sodium, pipotiazine, perphenazine, chlorpromazine, fluphenazine decanoate, thioridazine, sulpiride, penfluridol, clozapine, risperidone, olanzapine, clomipramine, amitriptyline, doxepin, fluoxetine, paroxetine, sertraline, fluvoxamine, citalopram, carbamazepine, sodium valproate, magnesium salicylate, sodium salicylate, diflunisal, salsalate, naproxen, fenbufen, sulindac, piroxicam, celecoxib, choline magnesium salicylate, acetaminophen, indomethacin, naproxen, nabumetone, diclofenac, nimesulide, fenbid, and voltaren. Example macromolecular drugs include a cancer vaccine, an anthrax vaccine, an influenza vaccine, a Lyme disease vaccine, a rabies vaccine, a measles vaccine, a mumps vaccine, a varicella vaccine, a smallpox vaccine, a hepatitis vaccine, a hepatitis A vaccine, a hepatitis B vaccine, a hepatitis C vaccine, a pertussis vaccine, a rubella vaccine, a diphtheria vaccine, an encephalitis vaccine, a Japanese encephalitis vaccine, a respiratory syncytial virus vaccine, a yellow fever vaccine, a polioencephalitis vaccine, a herpes vaccine, a human papillomavirus vaccine, a rotavirus vaccine, a pneumococcal vaccine, a meningitis vaccine, a pertussis vaccine, a tetanus vaccine, a typhoid vaccine, a cholera vaccines, a tuberculosis vaccine, a severe acute respiratory syndrome (SARS) vaccine, a HSV-1 vaccine, a HSV-2 vaccine, a HIV vaccine, and a combination thereof.

In the eleventh aspect, use of the soluble biomolecular glass microneedle is provided, where the soluble glass microneedle can be used to pierce skin and/or mucosa, blood-eye barrier, blood-brain barrier, and the like for pharmaceutical or aesthetic use.

The pharmaceutical use refers to a bioactive molecule transport platform, a biosensor, or a stimuli-responsive transport system.

The aesthetic use refers to use such as exfoliation or improving skin appearance, smoothness, or brightness.

In the present disclosure, terms such as “a,” “an,” and “the” do not merely refer to a single entity but rather encompass general categories, of which specific examples may be used for illustrations. The terms “a,” “an,” and “the” are used interchangeably with the term “at least one.” The phrases “at least one” and “including at least one” followed by a list refer to any one item in the list and any combination of two or more items in the list. Unless otherwise indicated, all numerical ranges include endpoints thereof and non-integer values between the endpoints.

In the present disclosure, the term “microneedle”, “microneedle array” or “array of microneedle” refers to a structure related to an array that can pierce the stratum corneum to facilitate transdermal delivery of a bioactive molecule to the skin. In many cases, they can be used interchangeably.

The biomolecular glass microneedle provided in the present disclosure has the following significant advantages:
(1) the biomolecular glass microneedle body in the present disclosure is formed by a therapeutic agent itself and is completely absorbable and bioavailable to the body without generating any metabolic burden;
(2) the therapeutic agent in the biomolecule glass microneedle of the present disclosure has high photostability and thermostability, facilitating long-term storage and use of the therapeutic agent;
(3) the microneedle in the present disclosure has a large loading capacity and enables rapid release of biomolecules, thereby effectively improving the bioavailability of the biomolecules; and
(4) the biomolecular glass microneedle in the present disclosure features a simple preparation process, is environmentally friendly with high batch repeatability, and is adapted to industrial production.

### BRIEF DESCRIPTION OF THE DRAWINGS

FIG. 1 is a photo of a thymopentin glass in Example 1;

FIG. 2 is a photo of a vancomycin glass in Example 2;

FIG. 3 is a photo of a soluble glass microneedle prepared in Example 3 (left) and a photo of a soluble glass microneedle prepared in Example 5 (right);

FIG. 4 is a photo of a soluble glass microneedle prepared in Example 4;

FIG. 5 is a photo of a soluble glass microneedle prepared in Example 6 (left) and a photo of a soluble glass microneedle prepared in Example 7 (right);

FIG. 6 is a photo of a soluble glass microneedle prepared in Example 8 (left) and a photo of a soluble glass microneedle prepared in Example 9 (right);

FIG. 7 is a scanning electron microscope photo of a soluble glass microneedle prepared in Example 10 (left) and a scanning electron microscope photo of a soluble glass microneedle prepared in Example 11 (right);

FIG. 8 is a microscope photo of a soluble glass microneedle prepared in Example 10 (left) and a microscope photo of a soluble glass microneedle prepared in Example 11 (right);

FIG. 9 is a photo of piercing a sealing film by a soluble glass microneedle prepared in Example 10;

FIG. 10 is a photo of piercing fresh pig ear skin by a soluble glass microneedle prepared in Example 1;

FIG. 11 is pharmacodynamic evaluation of a soluble thymopentin glass microneedle in Example 1 in rats;

FIG. 12 is pharmacodynamic evaluation on glucose control using a soluble insulin glass microneedle prepared in Example 9;

FIG. 13 is a time-varying curve of neurological scores of different groups in mice with experimental autoimmune encephalomyelitis (EAE); and

FIG. 14 is a release curve of a drug-loaded microneedle in Example 19 and a release curve of a drug-loaded microneedle in Example 20.

### DETAILED DESCRIPTION

The present disclosure is further detailed below with reference to the examples and drawings, but the embodiments of the present disclosure are not limited thereto.

### Microneedle Mold and Material Source

PDMS microneedle negative mold in the present disclosure is purchased from Taizhou Microchip Pharmaceutical Technology Co., Ltd. A functional portion of a microneedle array includes a conical type and a square pyramid type, with a height of approximately 300 or 600 microns and an aspect ratio of approximately 2:1. The microneedles are arranged in a square pattern consisting of approximately 10*10 microneedles with an equal spacing of 500 μm to 600 μm among the microneedles. Where no manufacturers are specified, the employed reagents or instruments are all conventional products commercially available through regular channels. Examples in which techniques or conditions are not specified are implemented based on the techniques or conditions described in the literature of the art or based on the product manuals.

In the following examples, the solvent content in the resulting glass is determined using a thermogravimetric analyzer.

### Preparation of Soluble Biomolecular Glass Microneedle

### Example 1

100 mg of thymopentin powder was weighed, and added to 10 mL of ultrapure water to obtain a thymopentin solution. After 20 mL of 0.01 M aqueous citric acid solution was added, a resulting solution system was measured to have a pH of 6.0, heated to a temperature of 50° C. at a heating rate of 10° C./min, and thermostatically kept at this temperature for 1 h to volatilize the solvent, thus obtaining a thymopentin glass when the solvent content was less than 2%.

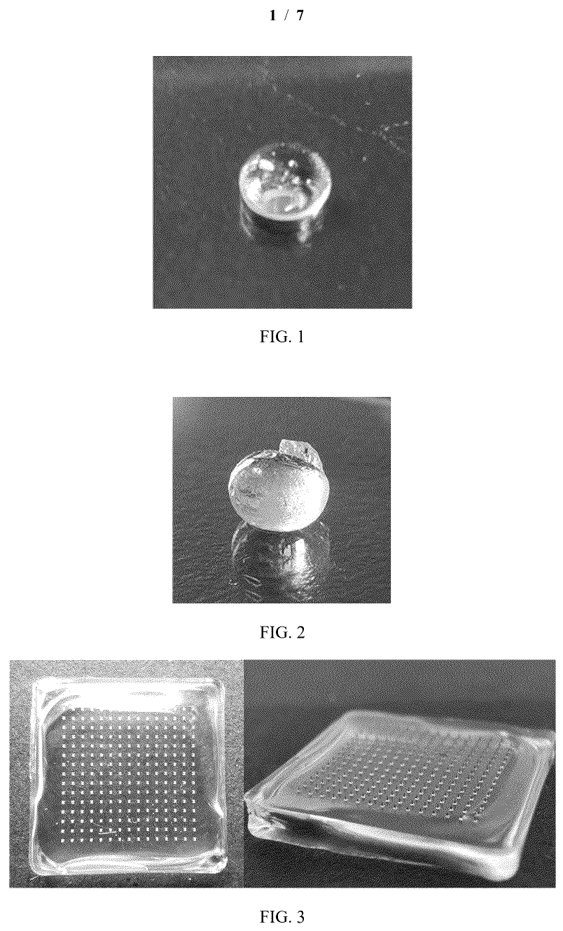

The resulting thymopentin glass is as shown in FIG. 1. As can be seen, the thymopentin glass exhibits glass properties, good transparency, tensile property, and processability.

The thymopentin glass was preheated at 60° C., and then poured into a PDMS microneedle negative mold, where needle tip and substrate portions of the microneedle mold were filled. After degassing by centrifugation at a speed of 3,000 rpm for 5 min, the mold was cooled. After the microneedle body was solidified, the microneedle glass was demolded, thus obtaining a soluble thymopentin glass microneedle.

### Example 2

100 mg of vancomycin powder was weighed, and added to 20 mL of ultrapure water and 1 mL of glycerol, to obtain a vancomycin solution. After 10 mL of 0.01 M aqueous hydrochloric acid solution was added, a resulting solution system was measured to have a pH of 4.0, heated to a temperature of 60° C. at a heating rate of 10° C./min, and thermostatically kept at this temperature for 3 h to volatilize the solvent, thus obtaining a vancomycin glass when the solvent content was less than 1%. The resulting vancomycin glass is as shown in FIG. 2.

The vancomycin glass was preheated at 60° C., and then poured into a PDMS microneedle negative mold, where needle tip and substrate portions of the microneedle mold were filled. After degassing by centrifugation at a speed of 2,000 rpm for 5 min, the mold was cooled. After the microneedle body was solidified, the microneedle glass was demolded, thus obtaining a soluble vancomycin glass microneedle.

### Example 3

500 mg of L-histidine powder was weighed, and added to 20 mL of ultrapure water and 1 mL of ethanol, to obtain a histidine solution. After 10 mL of 0.01 M aqueous hydrochloric acid solution was added, a resulting solution system was measured to have a pH of 5.0, heated to a temperature of 100° C. at a heating rate of 50° C./min, and thermostatically kept at this temperature for 0.5 h to volatilize the solvent, thus obtaining a histidine glass when the solvent content was less than 3%.

The arginine glass was preheated at 60° C., and then poured into a PDMS microneedle negative mold, where needle tip and substrate portions of the microneedle mold were filled. After rapid degassing under reduced pressure under a vacuum pump for 10 min, the mold was cooled to room temperature. After the microneedle body was solidified, the microneedle glass was demolded, thus obtaining a soluble histidine glass microneedle.

The resulting soluble histidine glass microneedle is as shown in FIG. 3 (left).

### Example 4

100 mg of aspartic acid powder was weighed, and added to 20 mL of ultrapure water and 1 mL of glycerol, to obtain an aspartic acid solution. After 10 mL of 0.01 M aqueous hydrochloric acid solution was added, a resulting solution system was measured to have a pH of 4.0, heated to a temperature of 60° C. at a heating rate of 10° C./min, and thermostatically kept at this temperature for 3 h to volatilize the solvent, thus obtaining an aspartic acid glass when the solvent content was less than 1%.

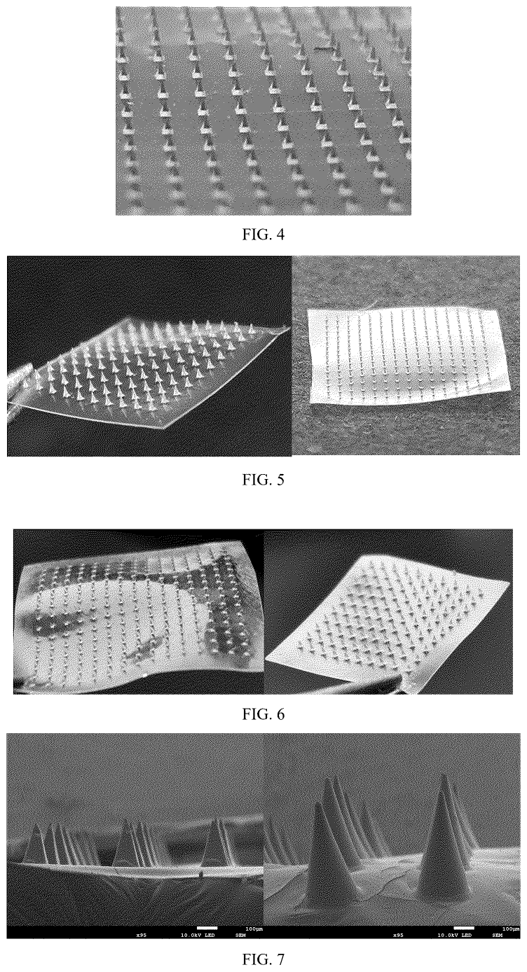

The aspartic acid glass was preheated at 80° C., and then poured into a PDMS microneedle negative mold, where needle tip and substrate portions of the microneedle mold were filled. After degassing by centrifugation at a speed of 2,000 rpm, the mold was cooled. After the microneedle body was solidified, the microneedle glass was demolded, thus obtaining a soluble aspartic acid glass microneedle. The resulting soluble aspartic acid glass microneedle is as shown in FIG. 4.

### Example 5

20 mg of tuftsin powder was weighed, and added to 20 mL of ultrapure water to obtain a tuftsin solution. After 10 mL of 0.01 M aqueous malic acid solution was added, a resulting solution system was measured to have a pH of 6.0, heated to a temperature of 60° C. at a heating rate of 10° C./min, and thermostatically kept at this temperature for 2 h to volatilize the solvent, thus obtaining a tuftsin glass when the solvent content was less than 1%.

The tuftsin glass was preheated at 50° C., and then poured into a PDMS microneedle negative mold. After degassing by centrifugation at a speed of 2,000 rpm for 5 min, the mold was cooled. After the microneedle body was solidified, the microneedle glass was demolded, thus obtaining a soluble tuftsin glass microneedle.

The resulting soluble histidine glass microneedle is as shown in FIG. 3 (right).

### Example 6

10 mg of semaglutide powder was weighed, and added to 20 mL of ultrapure water and 1 mL of glycerol, to obtain a semaglutide solution. After 10 mL of 0.01 M aqueous hydrochloric acid solution was added, a resulting solution system was measured to have a pH of 4.0, heated to a temperature of 60° C. at a heating rate of 10° C./min, and thermostatically kept at this temperature for 0.5 h to volatilize the solvent, thus obtaining a semaglutide glass when the solvent content was less than 1%.

The semaglutide glass was preheated at 60° C., and then filled in a PDMS microneedle negative mold by microfluidics, where merely a needle tip portion of the microneedle mold was filled. After degassing by centrifugation at a speed of 2,000 rpm for 5 min, the mold was cooled. After the needle tip portion of the microneedle was solidified, a 5% polyvinylpyrrolidone solution was poured into a substrate portion of the PDMS microneedle mold. After degassing by centrifugation at a speed of 2,000 rpm for 5 min, further, the mixture was dried at a temperature of 60° C. and a relative humidity of 50% for a duration of 6 h. After solidification, the microneedle glass was demolded, thus obtaining a soluble semaglutide glass microneedle.

The resulting soluble semaglutide glass microneedle is as shown in FIG. 5 (left).

### Example 7

20 mg of methotrexate powder was weighed, and added to 20 mL of ultrapure water, to obtain a methotrexate solution. After 10 mL of 0.01 M aqueous citric acid solution was added, a resulting solution system was measured to have a pH of 3.0, heated to a temperature of 60° C. at a heating rate of 10° C./min, and thermostatically kept at this temperature for 2 h to volatilize the solvent, thus obtaining a methotrexate glass when the solvent content was less than 1%.

The methotrexate glass was preheated at 50° C., and then poured into a PDMS microneedle negative mold, where merely a needle tip portion of the microneedle mold was filled. After degassing by centrifugation at a speed of 2,000 rpm for 5 min, the mold was cooled. After the needle tip portion of the microneedle was solidified, a 5% hyaluronic acid solution was poured into a substrate portion of the PDMS microneedle mold. After degassing by centrifugation at a speed of 2,000 rpm for 5 min, further, the mixture was dried at a temperature of 80° C. and a relative humidity of 30% for a duration of 6 h. After solidification, the microneedle glass was demolded, thus obtaining a soluble methotrexate glass microneedle.

The resulting soluble methotrexate glass microneedle is as shown in FIG. 5 (right).

### Example 8

20 mg of cyclosporin powder was weighed, and added to 20 mL of ultrapure water and 1 mL of ethanol, to obtain a cyclosporin solution. After 10 mL of 0.01 M aqueous lactobionic acid solution was added, a resulting solution system was measured to have a pH of 6.0, heated to a temperature of 100° C. at a heating rate of 10° C./min, and thermostatically kept at this temperature for 2 h to volatilize the solvent, thus obtaining a cyclosporin glass when the solvent content was less than 1%.

The cyclosporin glass was preheated at 120° C., and then poured into a PDMS microneedle negative mold, where merely a needle tip portion of the microneedle mold was filled. After degassing by centrifugation at a speed of 2,000 rpm for 5 min, the mold was cooled. After the needle tip portion of the microneedle was solidified, a 5% polyvinyl alcohol solution was poured into a substrate portion of the PDMS microneedle mold. After degassing by centrifugation at a speed of 2,000 rpm for 5 min, further, the mixture was dried at a temperature of 80° C. and a relative humidity of 50% for a duration of 6 h. After solidification, the microneedle glass was demolded, thus obtaining a soluble cyclosporin glass microneedle.

The resulting soluble cyclosporin glass microneedle is as shown in FIG. 6 (left).

### Example 9

10 mg of recombinant human insulin powder was weighed, and added to 20 mL of normal saline to obtain a recombinant human insulin solution. After 10 mL of 0.01 M aqueous adenosine-5′-monophosphate (AMP) solution was added, a resulting solution system was measured to have a pH of 7.0, heated to a temperature of 40° C. at a heating rate of 10° C./min, and thermostatically kept at this temperature for 6 h to volatilize the solvent, thus obtaining a recombinant human insulin glass when the solvent content was less than 1%.

The recombinant human insulin glass was preheated at 40° C., and then poured into a PDMS microneedle negative mold, where needle tip and substrate portions of the microneedle mold were filled. After degassing by centrifugation at a speed of 2,000 rpm, the mold was cooled. After the needle tip portion of the microneedle was solidified, a 5% hydroxypropylmethyl cellulose solution was poured into the substrate portion of the PDMS microneedle mold. After degassing by centrifugation at a speed of 2,000 rpm for 5 min, further, the mixture was dried at a temperature of 30° C. and a relative humidity of 1% for a duration of 12 h. After solidification, the microneedle glass was demolded, thus obtaining a soluble recombinant human insulin glass microneedle.

The resulting soluble recombinant human insulin glass microneedle is as shown in FIG. 6 (right).

### Example 10

100 mg of arginine powder was weighed, and added to 20 mL of ultrapure water, to obtain an arginine solution. After 10 mL of 0.01 M aqueous citric acid solution was added, a resulting solution system was measured to have a pH of 5.0, heated to a temperature of 120° C. at a heating rate of 10° C./min, and thermostatically kept at this temperature for 1 h to volatilize the solvent, thus obtaining an arginine glass when the solvent content was less than 1%.

The arginine glass was preheated at 120° C., and then poured into a PDMS microneedle negative mold, where needle tip and substrate portions of the microneedle mold were filled. After degassing by centrifugation at a speed of 2,000 rpm, the mold was cooled. After the microneedle body was solidified, the microneedle glass was demolded, thus obtaining a soluble arginine glass microneedle.

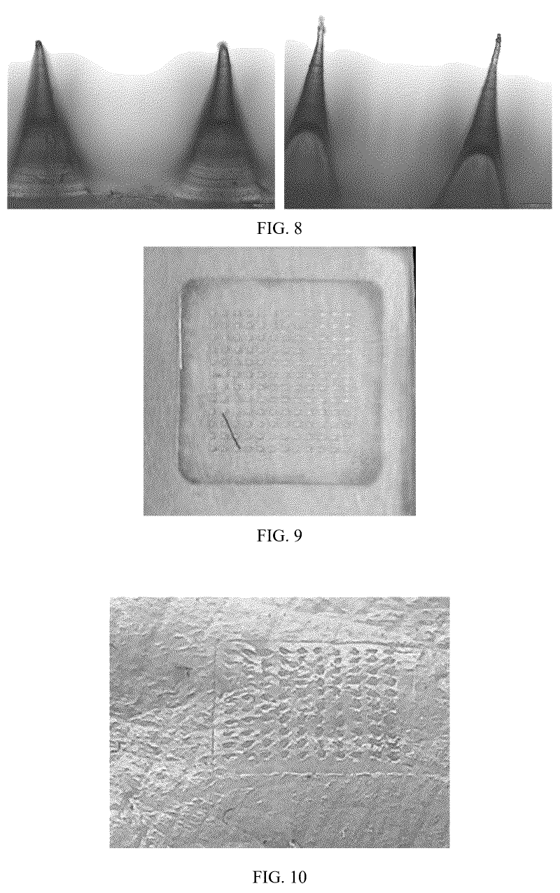

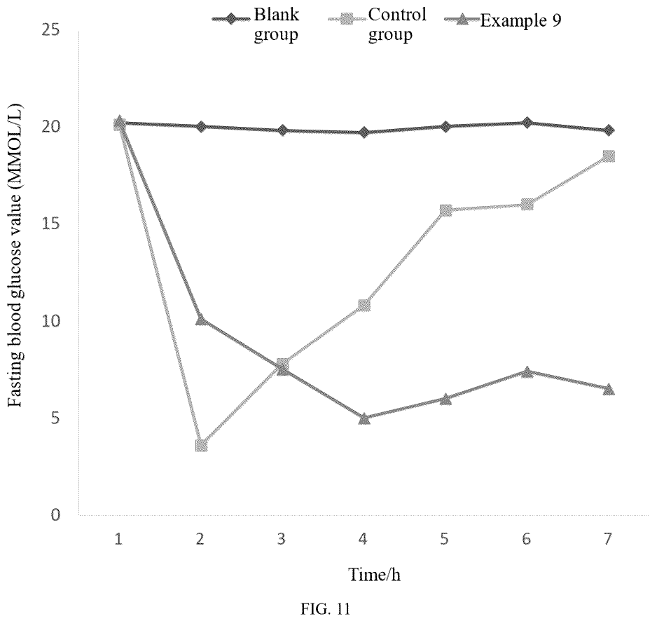

The resulting soluble arginine glass microneedle was observed macroscopically using a 3D microscope for its appearance, and microscopically using a scanning electron microscope (SEM) for its microneedle body, with the results as shown in FIG. 7 (left) and FIG. 8 (left).

### Example 11

The soluble arginine glass microneedle in the above Example 10 may also be prepared by combining a process of preparing a biomolecular glass by hydrothermal method with a process of preparing a microneedle by casting method, that is, prepared through the following steps:

100 mg of arginine powder was weighed, and added to 20 mL of ultrapure water, to obtain an arginine solution. After 10 mL of 0.01 M aqueous citric acid solution was added, a resulting solution system was measured to have a pH of 5.0.

The mixed arginine-citric acid precasting solution was poured into a PDMS microneedle mold, which was then heated to a temperature of 120° C. at a heating rate of 10° C./min, and thermostatically kept at this temperature for 1 h to volatilize the solvent. Then, after degassing by centrifugation at a speed of 2,000 rpm, the mold was cooled. After the microneedle body was solidified, the microneedle glass was demolded, thus obtaining a soluble arginine glass microneedle.

The resulting soluble arginine glass microneedle was observed macroscopically using a 3D microscope for its appearance, and microscopically using a scanning electron microscope (SEM) for its microneedle body, with the results as shown in FIG. 7 (right) and FIG. 8 (right).

As can be seen from the scanning electron microscope and optical microscope photos of the soluble arginine glass microneedle, a soluble glass microneedle with a complete needle tip can be obtained from the preparation method in each of Example 10 and Example 11. The macroscopic and microscopic images show that, regardless of a conical or square pyramidal needle shape, needle shape and needle tip sharpness of the microneedle are good and consistent with the PDMS mold design.

### Example 12: Elastic Modulus and Hardness of Soluble Biomolecular Glass Microneedle

A nanoindenter (Nano Indenter G200, Agilent) was used to measure the elastic modulus and the hardness of the soluble biomolecular glass microneedle. As can be seen from the result shown in Table 1, the soluble biomolecular glass microneedle prepared in each of Examples 1-11 has a high elastic modulus and a high hardness, laying the foundation for further application in skin penetration.

### Example 13: Experiment on Piercing Performance Evaluation of Soluble Biomolecular Glass Microneedle

The soluble microneedle was measured for piercing performance using a sealing film (Parafilm M Laboratory Film). 8 layers of the sealing film were affixed to a foam board to simulate artificial skin, which was pierced with the resulting soluble microneedle using an applicator. As can be seen from the result shown in FIG. 9, the sealing film was completely pierced, and the needle tip was not broken. The pierced sealing film was observed under a microscope for the number of holes, to calculate the piercing depth (depth=ΣD×a/A, where a is the number of holes; A is the total number of microneedles; and D is the thickness of a single sealing film layer, 125 μm). The result is listed in Table 1, where the needle tip length in Example 1, Example 2, and Example 11 is 600 μm, while the other glass microneedles have a needle tip length of 300 μm. The result shows that the soluble biomolecular glass microneedle in each example exhibits good piercing performance.

TABLE 1
Strength test and piercing depth of soluble
biomolecular glass microneedle

Needle tip parameters

| Example | Hardness | Elastic modulus | Piercing depth |
|---|---|---|---|
| No. | (Mpa) | (Gpa) | (μm) |
| Example 1 | 140 | 2.8 | 500 |
| Example 2 | 130 | 2.5 | 450 |
| Example 3 | 510 | 9.4 | 250 |
| Example 4 | 120 | 17 | 280 |
| Example 5 | 125 | 16 | 265 |
| Example 6 | 165 | 2.1 | 296 |
| Example 7 | 110 | 12 | 285 |
| Example 8 | 110 | 14 | 220 |
| Example 9 | 120 | 1.9 | 260 |
| Example 10 | 780 | 3.5 | 289 |
| Example 11 | 780 | 3.4 | 581 |

### Example 14: Experimental Method of Fresh Pig Skin Piercing

Fresh pig ear skin with a thickness of 800 μm was selected and pierced with the soluble microneedle prepared in Example 1 using an applicator, with the result as shown in FIG. 10. Clear needle holes of the microneedles are visible in the pig ear skin, and the number and arrangement of the needle holes were consistent with those in the microneedle array, showing that the microneedle has enough mechanical properties to pierce ex vivo pig skin.

### Example 15: Stability Evaluation of Biomolecule in Glass Microneedle

The microneedle array prepared in Example 9 was placed in a storage chamber maintained at 40° C. and 96% relative humidity (RH) along with an equivalent amount of an insulin solution. The insulin content was determined after storage in the chamber for 1, 3, 7, and 14 days. At the designated time points, the array was taken from the chamber and washed with 0.1 mol/L acetic acid (1 mL) to obtain the insulin solution. The insulin content in the resulting washing solution was analyzed by high-performance liquid chromatography (HPLC). The stability of insulin in the resulting microneedle was contrastively analyzed using the insulin solution under same storage conditions as the reference. The percentage of undegraded insulin remaining in the microneedle or solution at each time point was determined by measuring the insulin peak area in the selected sample and dividing it by the initially measured insulin peak area, with the result listed in Table 2.

| | Insulin content percentage | | | | |
|---|---|---|---|---|---|
| Sample | Initial | Day 1 | Day 3 | Day 7 | Day 14 |
| Solution control | 100 | 10 | 0 | 0 | 0 |
| Example 9 | 100 | 98 | 91 | 78 | 67 |

The above result shows that the insulin in the glass microneedle has significantly better stability than the solution group in the 14-day accelerated stability experiment, showing that the resulting soluble glass microneedle can effectively preserve the activity of bioactive molecules and facilitates long-term storage.

### Pharmacodynamic Evaluation of Biomolecule in Glass Microneedle

### Example 16: Pharmacodynamic Evaluation on Glucose Control Using a Soluble Recombinant Human Insulin Glass Microneedle Prepared in Example 9

Purchased spontaneous type I diabetic model mice (male, 8 weeks old) were divided into three groups, with 6 mice in each group. Mice in an experimental group were transdermally administered via a glass microneedle at a dose of 4 IU/Kg, mice in a blank group were not subjected to any treatment, and mice in a control group were injected with the recombinant insulin through the tail vein at a dose of 4 IU/Kg, to measure their blood glucose concentrations at different time points. Tail blood of the mice was taken every hour, to measure fasting blood glucose values of the mice with a glucometer. A curve was plotted with time as the abscissa, and the fasting blood glucose values of the experimental mice as the ordinate. The result is as shown in FIG. 11. As proven from a curve of blood glucose control in the mice with spontaneous type I diabetes, the soluble recombinant human insulin glass microneedle prepared in Example 9 can effectively control blood glucose in mice, and achieve more steady glucose control effects than injection insulin.

### Example 17: Pharmacodynamic Evaluation of Soluble Thymopentin Glass Microneedle in Rats

Experimental method: 28 SPF-grade male SD rats with body weights of 180-220 g without taking any other drugs before the experiment. Thymopentin powder was dissolved in normal saline to prepare a thymopentin solution for use as a reference formulation. The test formulation was the soluble thymopentin glass microneedle prepared in Example 1. 28 SD rats were randomly divided into 4 groups, named groups 1~4 respectively, with 7 rats in each group, for in vivo pharmacodynamic study.

An immunosuppression model was constructed before starting the experiment. An immunosuppressant cyclophosphamide powder was purchased from Jiangsu Hengrui Pharmaceuticals Co., Ltd and prepared into 1 mg/L cyclophosphamide solution with normal saline. Except for the group 1, rats in the other 3 groups were intraperitoneally injected with the immunosuppressant at a dose of 35 mg/(kg*d) for three consecutive days to suppress immune functions of the rats. The group 1 served as a blank control group, and was intraperitoneally injected with 35 mL/kg of normal saline.

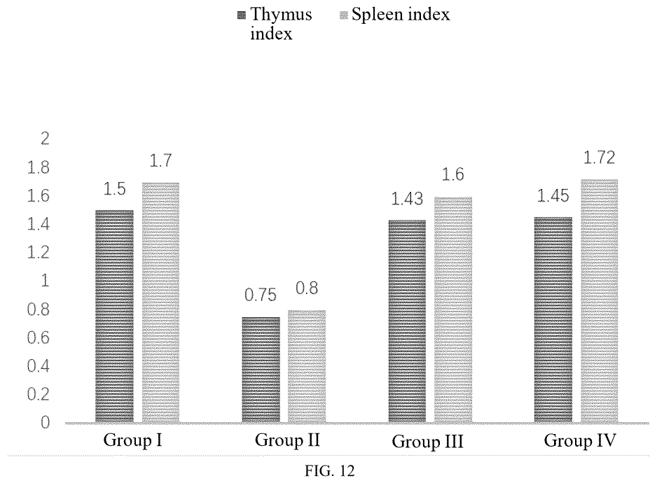

After the model was successfully constructed by injection for three consecutive days, pharmacodynamic evaluation was conducted. The rats in the group 1 served as the blank control group, the rats in the group 2 served as a negative control group, and they were all subcutaneously intravenously injected with 1 mL/kg of normal saline for seven consecutive days. The rats in the group 3 served as a positive control group, and were subcutaneously injected with the thymopentin solution at a dose of 100 μg/kg. The rats in the group 4 were treated with the soluble thymopentin glass microneedle prepared in Example 1 at a dose of 100 μg/kg for 7 consecutive days. Then, the rats in the 3 groups were killed by cervical dislocation, to collect and weigh the thymus and spleen of each rat, and calculate organ indexes as per the calculation formula below:

$$Organ index = \frac{{w}_{0}}{W}$$

where W₀ is mass of the thymus or spleen, and W is body weight of the rat. As shown in FIG. 12, the negative control group 2 has lower spleen and thymus indexes than the blank control group 1, proving that the immunosuppression model was successfully constructed. Each of the dosing groups 3 and 4 has higher spleen and thymus index values than the negative control group of the model group, the organ indexes of all immunosuppressed rats are improved to some extent, with statistically significant difference, proving that the soluble thymopentin glass microneedle has better pharmacodynamical effect than the solution dosage form.

### Example 18: Neurological Score of Microneedle Prepared in Example 5 in Mice with Experimental Autoimmune Encephalomyelitis (EAE)

Specifically, for the EAE group, 200 μg of MOG35-55 was dissolved in 200 μl of PBS buffer, the mixture was then fully mixed with 200 μl of Freund's complete adjuvant (CFA) to form an emulsion (final concentration of inactivated tuberculin bacillus was 5 mg/ml), and for the normal control group, 200 μl of PBS was directly fully mixed with 200 μl of CFA. 200 μg of the emulsion was subcutaneously injected at two sites on the back of the mice. 500 ng of pertussis toxin was intraperitoneally injected respectively in 0 h and 48 h after immunization. On day 1 after construction of the EAE model, the mice were transdermally administered on their backs via a glass microneedle at a dose of 100 μg/kg, for 7 consecutive days, to form a glass microneedle intervention group.

Neurological scores of experimental mice in each group: after immunization, the experimental mice in each group were evaluated for neurological scores every day and observed continuously for 30 days. Neurological scores were evaluated based on the following standards: 0 scores mean no symptoms; 1 score means reduced tail strain (complete weakness, tail tip unable to curl) or hindlimb weakness (unsteady gait); 2 scores mean tail and hindlimb weakness; 3 scores mean hindlimb paralysis (unilateral or bilateral hindlimbs can move to a certain extent); 4 scores mean complete hindlimb paralysis (hindlimbs are completely unable to move or are dragged by forelimbs to move); and 5 scores mean death.

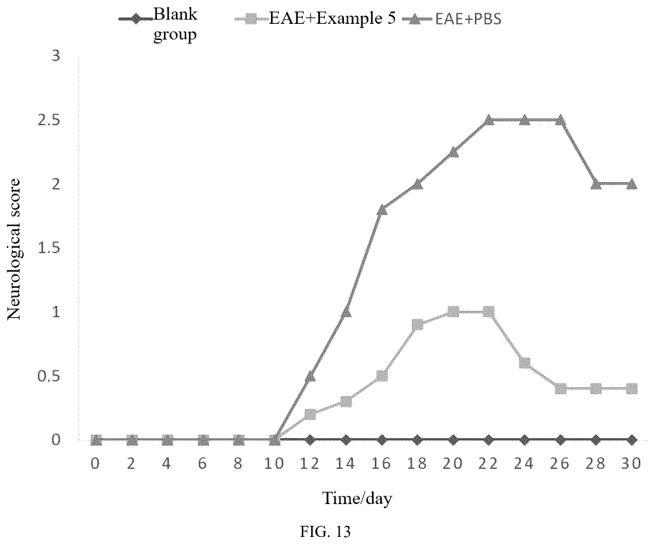

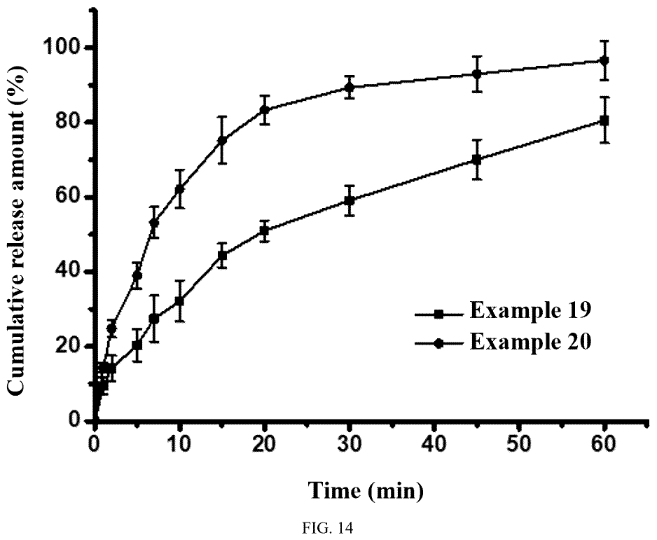

As indicated by the result of neurological scores in mice with experimental autoimmune encephalomyelitis (EAE) shown in FIG. 13, the soluble tuftsin glass microneedle prepared in Example 5 can effectively repair neurological functions in mice with experimental autoimmune encephalomyelitis (EAE).

### Example 19

As described in the tenth aspect, the microneedle provided in the present disclosure can also be loaded with an active drug for rapid and convenient transdermal drug delivery. Specifically, 100 mg of arginine powder and 0.5 mg of lidocaine hydrochloride powder were weighed, and added to 20 mL of ultrapure water, to obtain an arginine-lidocaine solution. After 10 mL of 0.01 M aqueous citric acid solution was added, a resulting solution system was measured to have a pH of 5.0.

The mixed casting solution was poured into a PDMS microneedle mold, which was then heated to a temperature of 40° C. at a heating rate of 10° C./min, and thermostatically kept at this temperature for 12 h to volatilize the solvent. Then, after degassing by centrifugation at a speed of 2,000 rpm, the mold was cooled. After the microneedle body was solidified, the microneedle glass was demolded, thus obtaining a soluble arginine-lidocaine glass microneedle.

### Example 20

100 mg of arginine powder and 0.2 mg of loratadine were weighed, and added to 20 mL of ultrapure water, to obtain an arginine-loratadine solution. After 10 mL of 0.01 M aqueous citric acid solution was added, a resulting solution system was measured to have a pH of 5.0.

The mixed casting solution was poured into a PDMS microneedle mold, which was then heated to a temperature of 40° C. at a heating rate of 10° C./min, and thermostatically kept at this temperature for 12 h to volatilize the solvent. Then, after degassing by centrifugation at a speed of 2,000 rpm, the mold was cooled. After the microneedle body was solidified, the microneedle glass was demolded, thus obtaining a soluble arginine-loratadine glass microneedle.

### Example 21: Measurement of Release Curve of Drug-Loaded Soluble Microneedle

Kinetic characteristics of in vitro drug release of the resulting drug-loaded microneedle were measured by encapsulating the drug-loaded microneedle prepared in each of the above Example 19 and Example 20 in a dialysis bag (molecular weight cutoff: 3 kDa), and then soaking the bag in a phosphate buffer solution (PBS, pH=7.4) at 37° C., where the drug-loaded microneedle was dissolved in the dialysis bag, so that the drug was released into the PBS through the dialysis bag. Samples were collected at preset time points (0.2 mL per sampling), and an equal amount of the PBS solution was supplemented to measure a cumulative drug release rate of the microneedle. As can be seen from the result shown in FIG. 14, the cumulative release amount of the drug exceeds 80% within 1 hour, showing that the microneedle can quickly release the loaded drug.

The applicant declares that the present disclosure shows the detailed method of the present disclosure with reference to the above embodiments, but the present disclosure is not limited to the above detailed method, which does not mean that the present disclosure can only be implemented by relying on the above detailed method. Those skilled in the art should understand that any improvement of the present disclosure, the equivalent replacement of each raw material of the product of the present disclosure, the addition of auxiliary components, the selection of specific methods, and the like are all encompassed within the scope of protection and the scope of disclosure of the present disclosure.

What is claimed is:

1. A biomolecular glass microneedle, comprising a microneedle array and a substrate;
wherein at least a part of the microneedle array consists of a biomolecular glass;
the biomolecular glass consists of a biomolecule, an inducer, and a solvent in a trace amount; the biomolecule is an amino acid and a derivative and/or a peptide thereof;
the solvent in the trace amount means that a content of the solvent in the biomolecular glass is 0.1 wt % to 5 wt %; the inducer is selected from the group consisting of a nucleotide, a nucleotide polymer, RNA, DNA, and/or a pH regulator; and the pH regulator is one or more selected from the group consisting of DL-tartaric acid, hydrochloric acid, sulfuric acid, phosphoric acid, lactic acid, lactobionic acid, citric acid, tartaric acid, oxalic acid, DL-malic acid, maleic acid, quinic acid, adipic acid, fumaric acid, hexanoic acid, heptanoic acid, caprylic acid, valeric acid, butyric acid, propionic acid, and glacial acetic acid.

2. The biomolecular glass microneedle according to claim 1, wherein a ratio of Tg (glass transition temperature)/Tm (melting temperature) of the biomolecular glass ranges from 0.55 to 0.75.

3. The biomolecular glass microneedle according to claim 1, wherein a ratio of Tg (glass transition temperature)/Tm (melting temperature) of the biomolecular glass ranges from 0.66 to 0.75.

4. The biomolecular glass microneedle according to claim 1, wherein the solvent is water, normal saline, or a mixed solvent of water and ethanol, or a mixed solvent of water and glycerol, and a proportion of solvents other than water in the mixed solvent is 0 to 50 v/v %.

5. The biomolecular glass microneedle according to claim 1, wherein the biomolecular glass microneedle has a hardness greater than 50 MPa;
and the biomolecular glass microneedle has an elastic modulus greater than 1 Gpa.

6. The biomolecular glass microneedle according to claim 1, wherein the substrate of the soluble biomolecular glass microneedle is made from a biomolecular glass and/or a polymer.

7. A preparation method of the biomolecular glass microneedle according to claim 1, comprising steps of:
(1) preparing the biomolecular glass by hydrothermal method: dissolving a certain amount of a biomolecular raw material in water or a mixed solvent, adding an appropriate amount of an inducer, adjusting pH of a resulting solution system;
maintaining the system at a constant temperature by hydrothermal method, and adjusting a duration of the constant temperature to volatilize the solvent stepwise, so as to obtain the biomolecular glass;
(2) preheating the biomolecular glass, then preparing a microneedle array by casting method, stretching method, atomization spraying method, microfluidics, or 3D printing; and
(3) finally, casting and degassing on the basis of the microneedle array to form a microneedle substrate.

8. The preparation method of the biomolecular glass microneedle according to claim 7, wherein, in the step of preparing the biomolecular glass by hydrothermal method, the system is adjusted to a pH of 1 to 9.

9. The preparation method of the soluble biomolecular glass microneedle according to claim 7, wherein the casting method comprises self-leveling, high-pressure injection, and microfluidics; and a method of the degassing comprises centrifugation, depressurization, or vacuum adsorption.

10. A method of piercing skin and/or mucosa, blood-eye barrier, blood-brain barrier, comprising step of using the biomolecular glass microneedle according to claim 1 to pierce skin, mucosa, blood-eye barrier, or blood-brain barrier.

11. The biomolecular glass microneedle according to claim 1, wherein a content of the solvent in the biomolecular glass is 0.1 wt % to 2 wt %.

12. The biomolecular glass microneedle according to claim 4, wherein a proportion of solvents other than water in the mixed solvent is 5 v/v % to 10 v/v %.

13. The preparation method of the biomolecular glass microneedle according to claim 7, wherein, in the step of preparing the biomolecular glass by hydrothermal method, the system is adjusted to a pH of 3 to 7.

14. The preparation method of the biomolecular glass microneedle according to claim 7, wherein the constant temperature is 20° C. to 120° C.

15. The preparation method of the biomolecular glass microneedle according to claim 7, wherein the constant temperature is 40° C. to 100° C.

16. The preparation method of the biomolecular glass microneedle according to claim 7, wherein the duration of the constant temperature is 5 min to 6 h.

17. The preparation method of the biomolecular glass microneedle according to claim 7, wherein the duration of the constant temperature is 30 min to 3 h.

18. The preparation method of the biomolecular glass microneedle according to claim 7, wherein a preheating temperature is 20° C. to 200° C.

19. The preparation method of the biomolecular glass microneedle according to claim 7, wherein a preheating temperature is 40° C. to 160° C.

20. The preparation method of the biomolecular glass microneedle according to claim 7, wherein a preheating temperature is 40° C. to 100° C.
